(19) United States
(12) Patent Application Publication
(10) Pub. No.: US 2025/0303089 A1
(43) Pub. Date: Oct. 2, 2025

(54) SURGICAL CRICOTHYROTOMY DEVICE

(71) Applicant: Intermed Labs LLC, Morgantown, WV (US)

(72) Inventors: Justin R. Chambers, Morgantown, WV (US); William T. McClellan, Morgantown, WV (US); Kyle Davis, Morgantown, WV (US)

(21) Appl. No.: 19/097,252

(22) Filed: Apr. 1, 2025

Related U.S. Application Data
(60) Provisional application No. 63/573,134, filed on Apr. 2, 2024.

(51) Int. Cl. A61M 16/04

(52) U.S. Cl. CPC A61M 16/0472; A61M 2205/586

(57) ABSTRACT

A cricothyrotomy device includes: a scalpel portion comprising a blade at a first end of the device; and a tracheal hook portion comprising a tracheal hook at a second end of the device opposite the first end of the device.

### PRIORITY

This application claims priority to U.S. provisional patent application No. 63/573,134 filed Apr. 2, 2024, the contents of which are incorporated by reference herein in their entirety.

### FIELD OF THE INVENTION

Aspects of the present invention relate generally to medical devices and, more particularly, to devices and methods for performing a cricothyrotomy.

### BACKGROUND

Cricothyrotomy (also called cricothyroidotomy) is a surgical airway procedure wherein an airway device is inserted by a medical professional into the patient's trachea through an incision made in the cricothyroid membrane. The cricothyroid membrane is made of elastic tissue located inferior to the thyroid cartilage and superior to the cricoid cartilage. The technique is used by medical professionals to establish an airway for oxygenation and ventilation of a patient. In certain situations, this technique is a lifesaving procedure when other techniques such as intubation have been or will likely be unsuccessful. After making an incision, the medical professional inserts a tube through the incision in the cricothyroid membrane to allow an emergency airway into the neck of the patient.

When performed an emergency cricothyrotomy can be a lifesaving procedure for a patient with a failed airway. A patient who is not properly oxygenated has a reduced chance of survival. The long-term damage to the patient is amplified over time. Cricothyroidotomy is often used in hostile and dynamic environments as might be found in military combat or motor vehicle accidents. A medical professional will consider using the procedure to ventilate and oxygenate a patient's lungs when there is a massive hemorrhage, profound vomiting, obstructing lesions, traumatic or congenital deformities, swelling, or other problems.

Surgical airway procedures have persisted as viable medical treatments from the late 1800's when surgeons utilized the techniques during anesthesia for amputation procedures. The biggest application is within emergency medicine to compensate for respiratory depression.

### SUMMARY

Various surgical tools have been designed to accommodate different steps in the surgical procedure. Some designs were created to incorporate a scalpel blade with a blunt dissection tool. Some devices included curved Kelly forceps incorporated with a blade scalpel. Others utilized a retracting scalpel blade and/or a retracting tracheal hook. These devices use springs, actuators, and sliding mechanisms to deploy the scalpel and/or the tracheal hook, but their performance is greatly diminished by debris, such as dirt and sand, and fluids, like blood or other bodily fluids. A failure to deploy under adverse circumstances can lead to adverse patient outcomes. Many other prior devices consist of kits that include multiple separate devices in a sealed bag or container and are awkward to utilize quickly.

Since the need to address airway obstruction is critical for the survival of patients, and the procedures are performed in high stress settings, the medical professional needs a device that is easy to use and incorporates tools that can perform multiple steps of the procedure. It is important to avoid the need of multiple separate tools to perform the procedure in high stress environments. It is equally critical that a device functions every time without risk of jamming or gumming up in adverse environments containing debris and fluids.

Various implementations of the invention address the above-noted problems of the prior art by providing an inventive device that combines a multitude of different devices with the scalpel blade to eliminate steps and expedite the surgical procedure. Devices in accordance with aspects of the present disclosure satisfy the needs of the patient and medical professional to improve outcomes by relying less on mechanical actuation and more on the manipulation by the operator. In embodiments, a tracheal hook and a scalpel blade are oriented at opposite poles of the device with high friction ergonomic grips to prevent loss of control by the user. Devices in accordance with aspects of the present disclosure minimize the incision and the manipulation needed to insert a successful surgical airway tube. Devices in accordance with aspects of the present disclosure enable a one-for-one transition of the scalpel blade with the tracheal hook into the trachea without increasing the likelihood of operator error or failure due to environmental problems. Embodiments of the devices may be made from biocompatible materials and are uniquely designed for simplicity.

When properly educated and trained, medical professionals of all types and other non-medical personnel can perform the cricothyrotomy procedure using various ones of the devices according to the present disclosure. Personnel who may be trained to perform the procedure with various ones of the devices include surgeons, combat medics, first responders, nurses, and possibly soldiers or other non-medical personnel.

The more convoluted the surgical device the more time is consumed in performance of the procedure increasing adverse outcomes for the patient. A surgical airway device advantageously should accommodate the dexterity of the medical professional and require minimal user movement.

The innovative surgical cricothyrotomy devices disclosed herein are an improvement over existing devices and allow medical professionals to perform emergency cricothyrotomy with greater efficiency for improved outcomes. The disclosed devices are configured to be used by medical professionals on adult patients, are maneuverable, bio-compatible, mechanically strong, ergonomic, re-usable, and can be manufactured and sold inexpensively.

In accordance with an aspect of the invention, a cricothyrotomy device includes: a scalpel portion comprising a blade at a first end of the device; and a tracheal hook portion comprising a tracheal hook at a second end of the device opposite the first end of the device.

### BRIEF DESCRIPTION OF THE SEVERAL VIEWS OF THE DRAWINGS

Aspects of the present invention are described in the detailed description which follows, in reference to the noted plurality of drawings by way of non-limiting examples of exemplary embodiments of the present invention. In the drawings, the same reference characters may refer to the same or similar parts throughout the different views.

FIGS. 1A and 1B show a cricothyrotomy device in accordance with aspects of the present invention.

FIG. 2 shows a cricothyrotomy device in accordance with aspects of the present invention.

FIG. 3 shows a cricothyrotomy device in accordance with aspects of the present invention.

FIGS. 4A-4C show a cricothyrotomy device in accordance with aspects of the present invention.

FIGS. 5A-5D show a cricothyrotomy device in accordance with aspects of the present invention.

FIGS. 6A-6D show a cricothyrotomy device in accordance with aspects of the present invention.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details in more detail than is necessary for the fundamental understanding of aspects of the present invention, the description taken with the drawings making apparent to those skilled in the art how several forms of the present invention may be embodied in practice.

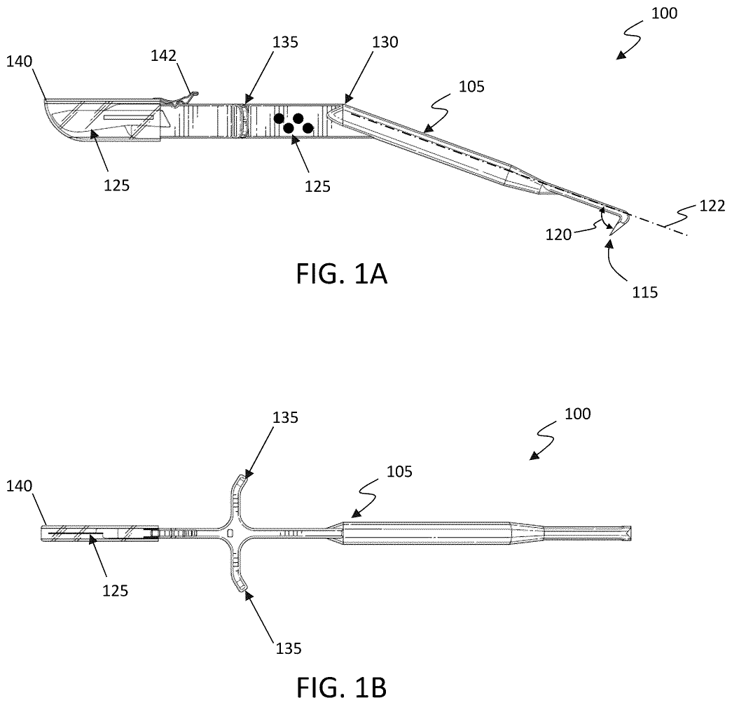

FIGS. 1A and 1B show a cricothyrotomy device 100 in accordance with aspects of the present invention. In embodiments, the device 100 includes a body 105 comprising a scalpel portion comprising a blade 110 at a first end of the body 105 and a tracheal hook portion comprising a tracheal hook 115 at a second end of the body 105 opposite the first end of the body 105.

In embodiments, the tracheal hook 115 comprises a structural element that forms an acute angle 120 with a longitudinal axis 122 associated with the second end of the body 105. The tracheal hook 115 may be integrally formed with the body 105, e.g., by manufacturing the body 105 and the tracheal hook 115 together as a unitary structure such as by molding or additive manufacturing.

In various embodiments, the body 105 is ergonomically designed for ease of manipulation by the user and may be composed of high friction materials and surface geometries to prevent the sliding of the medical professional's hands during a procedure, even in adverse environments. In some configurations, the body 105 includes one or more gripping features that are configured to enhance a user's grip of the device 100 when the user is holding and utilizing the device 100 to perform a cricothyrotomy procedure on a patient (e.g., an injured person in need of an emergency airway). The gripping features may include, for example and without limitation, one or more of a surface texturing 125 on the body 105, a bend 130 in the body 105, or one or more grip tabs 135 extending outward from the body 105. Surface texturing 125 may include knurling or other forms of physical surface texturing that provide a textured (e.g., uneven) exterior surface on at least a portion of the body 105. Grip tabs 135 may include one or more shapes that protrude outward from the body and that are configured to be contacted by one or more fingers (and/or thumb) of a user when the user is manipulating the device 100. The size, geometry, location, and quantity of grip tabs 135 may vary in different implementations. A bend 130 may comprise an angle or curve formed in the body 105, e.g., at an intersection of two portions of the body 105 at or near a midsection of the body 105 between the blade 110 and the tracheal hook 115, to provide greater control and manipulation for the user. If one or more grip tabs 135 are present, then surface texturing 125 may be provided at any desired location including on the grip tabs 135 and/or on other portions of the body 105 apart from the grip tabs 135.

The body 105, including any gripping features, may be composed of high friction material types, such as medical grade polymers or rubbers, to enhance the user's grip on the device 100. Gripping features, if present in the device 100, may be integrally formed with the body 105, e.g., by manufacturing the body 105 and gripping features together as a unitary structure such as by molding or additive manufacturing.

With continued reference to FIGS. 1A and 1B, the blade 110 may comprise a scalpel blade, for example. According to aspects of the present invention, the blade 110 is located at or proximate to the opposite end of the device from the tracheal hook 115 and is made from high strength material with a sharpened edge able to make one or more fine incisions in the patient. The blade 110 can be made from any conventional or later-developed material(s), such as corrosion resistant medical grade stainless steel with a chromium oxide coating, for example and without limitation.

In one exemplary implementation, the blade 110 is removably attached to the body 105 so that the blade 110 can be used and then removed and replaced with a new blade. In another exemplary implementation, the blade 110 is integrally (e.g., permanently) connected to the body 105, e.g., by molding or printing the body 105 around the blade 110, such that the blade 110 cannot be disconnected from the body without breaking either the blade 110 or the body 105.

The device 100 may include a blade cover 140 that is used to protect the blade 110 from damage during storage and use and to protect the user, patient, or other personnel from accidental and harmful incisions. In some embodiments, the blade cover 140 is removably attached to the body 105 and may be removed and discarded (or set aside) prior to using the device 100 to perform a cricothyrotomy procedure on a patient. In other embodiments, the blade cover 140 is movably attached to the body 105 through an attachment means that is configured to be articulated by the user to expose the blade 110. In one example, the attachment means comprises blade cover 140 having rails that mate with sliding rails on the body 105. This allows the blade cover 140 to slide back and forth relative to the body 105. In this example a tab 142 acts as a locking feature and allows it to lock in two positions, open and closed, and a user may push on the tab 142 to release and move the blade cover 140 to the opposite position. The blade cover 140 may include a blade cover latching means that is configured to secure the blade cover 140 over the blade 110 when not in use, and in some embodiments, to secure the blade cover 140 to the body 105 when articulated by the user to expose the blade 110. In the example described above, the blade cover latching means may comprise the tab 142 being configured to fall into locking grooves in the body 105 at each open and closed position of the blade cover 140. In this example, pushing the tab 142 releases it from the locking groove

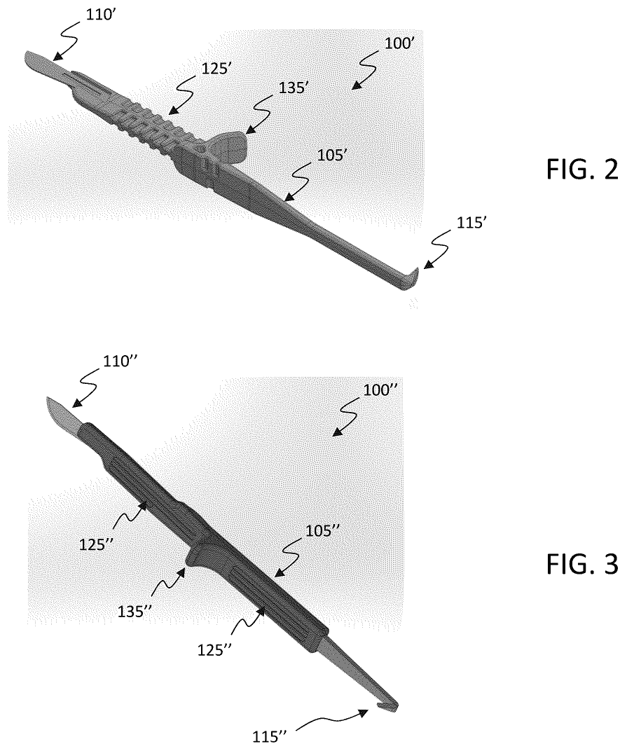

FIG. 2 shows a cricothyrotomy device 100′ in accordance with aspects of the present invention. In the configuration shown in FIG. 2, the device 100′ includes a body 105′, blade 110′, tracheal hook 115′, surface texturing 125′, and grip tab 135′ with the body 105′ having a straight-line center axis from the tracheal hook 115′ to the blade 110′ (e.g., no bend). In this example, the body 105′, tracheal hook 115′, surface texturing 125′, and grip tab 135′ are composed of a same material, and may be manufactured as a unitary structure by molding, additive manufacturing, or the like. In this example, the blade 110′ is removably attached to the body 105′ so that the blade 110′ can be used and then removed and replaced with a new blade. As shown in FIG. 2, and in accordance with aspects of the present invention, the tracheal hook 115′ and the grip tab 135′ each protrude outward from the same side of the body 105′, which advantageously enhances the user's grip when utilizing the tracheal hook 115′ to manipulate the opening formed in the patient's tissue before inserting the airway tubing.

FIG. 3 shows a cricothyrotomy device 100″ in accordance with aspects of the present invention. In the configuration shown in FIG. 3, the device 100″ includes a body 105″, blade 110″, tracheal hook 115″, surface texturing 125″, and grip tab 135″ with the body 105″ having a straight-line center axis from the tracheal hook 115″ to the blade 110″ (e.g., no bend). In this example, the body 105″, surface texturing 125″, and grip tab 135″ are composed of a same material, and may be manufactured as a unitary structure by molding, additive manufacturing, or the like. In the configuration shown in FIG. 3, the blade 110″ and the tracheal hook 115″ are each composed of a material different than the material of the body 105″ and are permanently connected to the body 105″. For example, the blade 110″ and the tracheal hook 115″ may each be composed of metal and the body 105″ may be composed of a plastic that is formed in a permanent connection around the metal of each of the blade 110″ and the tracheal hook 115″. As shown in FIG. 3, and in accordance with aspects of the present invention, the tracheal hook 115″ and the grip tab 135″ each protrude outward from the same side of the body 105″. As shown in FIG. 3, the surface texturing 125″ includes multiple parallel ridges extending longitudinally along different portions of the body 105″ on the same side of the body 105″ from which the tracheal hook 115″ and the grip tab 135″ protrude outward. Although not shown in FIG. 3, the side of the body 105″ opposite the side from which the tracheal hook 115″ and the grip tab 135″ protrude outward may include surface texturing similar to surface texturing 125″.

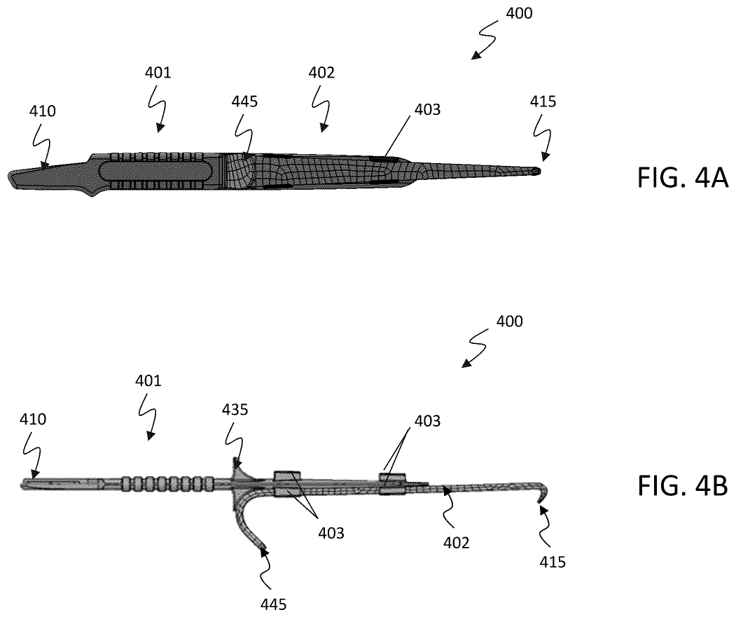

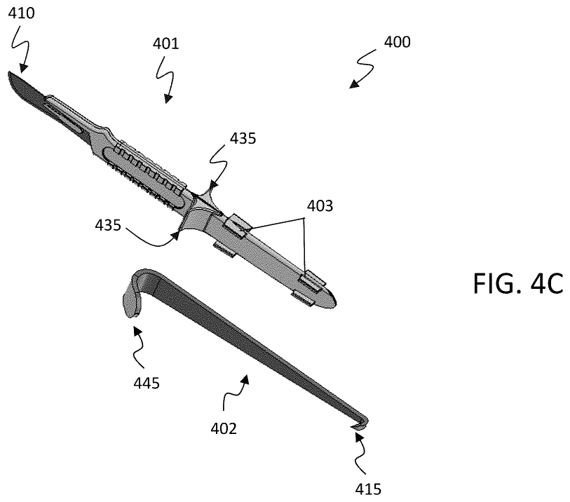

FIGS. 4A, 4B, and 4C show a cricothyrotomy device 400 in accordance with aspects of the present invention. In embodiments, the device 400 includes a scalpel portion 401 and a tracheal hook portion 402 that are releasably connected to one another, e.g., by one or more resilient clips 403. In embodiments, the scalpel portion 401 includes a blade 410 (which may be similar of similar construction and function as blade 110) and the tracheal hook portion 402 includes a tracheal hook 415 (which may be similar of similar construction and function as tracheal hook 115). As shown in FIGS. 4A-C, the scalpel portion 401 may include one or more gripping features such as surface texturing 425 (which may be similar of similar construction and function as surface texturing 125) and grip tabs 435 (which may be similar of similar construction and function as grip tab 135), and the tracheal hook portion 402 may include one or more gripping features such as grip tab 445 (which may be similar of similar construction and function as grip tab 135).

In accordance with aspects of the present invention, the device 400 is sized and shaped such that when the scalpel portion 401 and a tracheal hook portion 402 are connected to one another, e.g., by the one or more resilient clips 403, the blade 410 is at a first end of the device 400 and the tracheal hook 415 is at a second end of the device 400 opposite the first end of the device 400. The device 400 may be used in the connected configuration, e.g., in a manner similar to the device 100 of FIGS. 1A and 1B. Alternatively, the user may disconnect the scalpel portion 401 and a tracheal hook portion 402 from one another and use them separately, e.g., as shown in FIG. 4C. This provides an advantage of enabling the user to use either configuration depending on their preference or as dictated by the needs of the situation.

In accordance with aspects of the present invention, the device 400 is sized and shaped such that the grip tab 445 of the tracheal hook portion 402 abuts a grip tab 435 of the scalpel portion 401 when the scalpel portion 401 and the tracheal hook portion 402 are connected to one another, e.g., as shown in FIG. 4B. In accordance with aspects of the present invention, the device 400 is sized and shaped such that a flat surface of the tracheal hook portion 402 abuts a flat surface of the scalpel portion 401 when the scalpel portion 401 and the tracheal hook portion 402 are connected to one another, e.g., as shown in FIG. 4B.

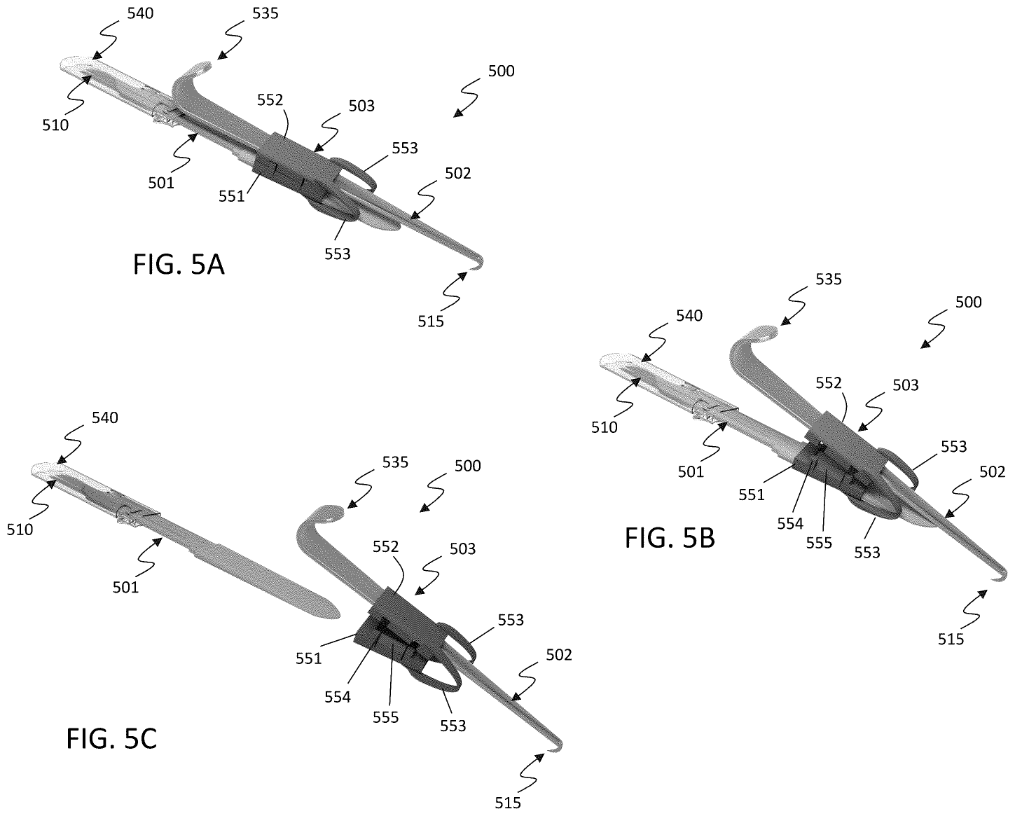

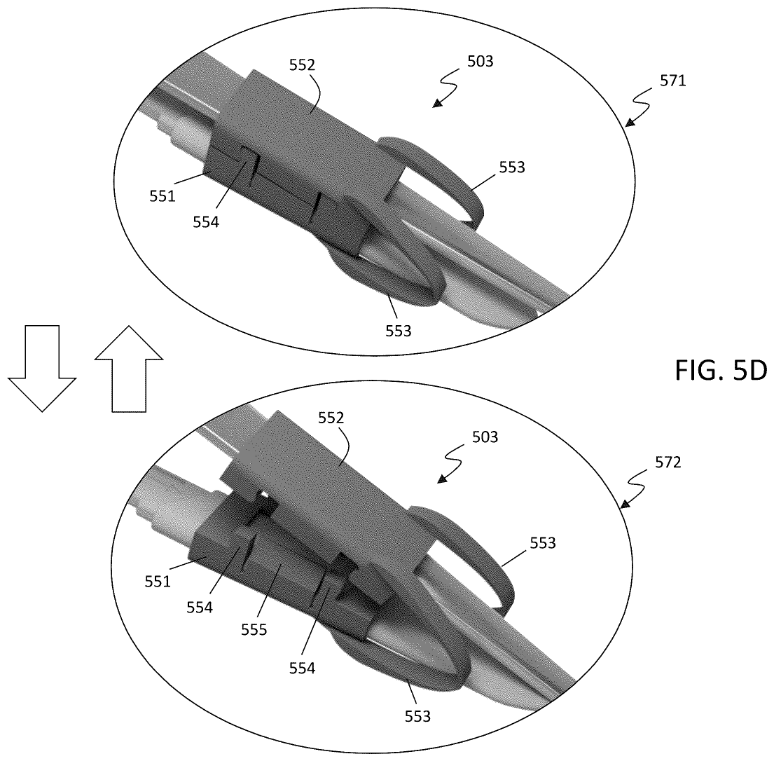

FIGS. 5A-5C show a cricothyrotomy device 500 in accordance with aspects of the present invention. In embodiments, the device 500 includes a scalpel portion 501 and a tracheal hook portion 502 that are releasably connected to one another, e.g., by a locking mechanism 503. In embodiments, the scalpel portion 501 includes a blade 510 (which may be similar of similar construction and function as blade 110) and the tracheal hook portion 502 includes a tracheal hook 515 (which may be similar of similar construction and function as tracheal hook 115). Each of the scalpel portion 501 and the tracheal hook portion 502 may include one or more gripping features such as surface texturing, one or more grip tabs, etc., e.g., as described herein. For example, as shown in FIGS. 5A-5C, the tracheal hook portion 502 may comprise a grip tab 535. The scalpel portion 501 may include a blade cover 540 (which may be similar of similar construction and function as blade cover 140).

In accordance with aspects of the present invention, the device 500 is sized and shaped such that when the scalpel portion 501 and a tracheal hook portion 502 are connected to one another by the locking mechanism 503, the blade 510 is at a first end of the device 500 and the tracheal hook 515 is at a second end of the device 500 opposite the first end of the device 500. The device 500 may be used in the connected configuration shown in FIG. 5A, e.g., in a manner similar to the device 100 of FIGS. 1A and 1B. Alternatively, the user may disconnect the scalpel portion 501 from the tracheal hook portion 502 and use them separately, e.g., as shown in FIG. 5C. This provides an advantage of enabling the user to use either configuration (e.g., connected or disconnected) depending on their preference or as dictated by the needs of the situation.

In accordance with aspects of the present invention, the locking mechanism 503 is configured to lock the scalpel portion 501 into place relative to the tracheal hook portion 502 when the locking mechanism 503 is engaged, and to permit separating the scalpel portion 501 from the tracheal hook portion 502 when the locking mechanism 503 is disengaged. In embodiments, the locking mechanism 503 comprises a first portion 551 connected to the scalpel portion 501 and a second portion 552 connected to the tracheal hook portion 502. The first portion 551 and the second portion 552 may be permanently connected to one another by a hinge such as a living hinge 553. In embodiments, the first portion 551 and the second portion 552 are releasably connected to one another by a snap mechanism 554 that engages when the first portion 551 and the second portion 552 are pressed against one another. In embodiments, one of the first portion 551 or the second portion 552 includes a wedge 555 that creates a friction lock amongst the scalpel portion 501, the tracheal hook portion 502, and the locking mechanism 503 when the first portion 551 and the second portion 552 are pressed against one another to engage the snap mechanism 554, e.g., as shown in FIG. 5A. In embodiments, when the first portion 551 and the second portion 552 are moved apart from one another in a manner that disengages the snap mechanism 554, e.g., as shown in FIG. 5B, this relative movement between the first portion 551 and the second portion 552 disengages the friction lock, such that the scalpel portion 501 can be removed from the locking mechanism, e.g., as shown in FIG. 5C. The locking mechanism 503 may be configured such that the scalpel portion 501 is removable (as shown in FIG. 5C) or alternatively such that the tracheal hook portion 502 is removable. FIG. 5D illustrates a first position 571 of the locking mechanism 503 in which the first portion 551 and the second portion 552 are pressed against one another to engage the snap mechanism 554, e.g., as shown in FIG. 5A, and a second position 572 of the locking mechanism 503 in which the first portion 551 and the second portion 552 are moved apart from one another in a manner that disengages the snap mechanism 554, e.g., as shown in FIG. 5B. In one example, and as depicted in FIG. 5D, the snap mechanism 554 may comprise one or more pairs of mating features, such as a post and a recesses having corresponding shapes, on different ones of the first portion 551 and the second portion 552.

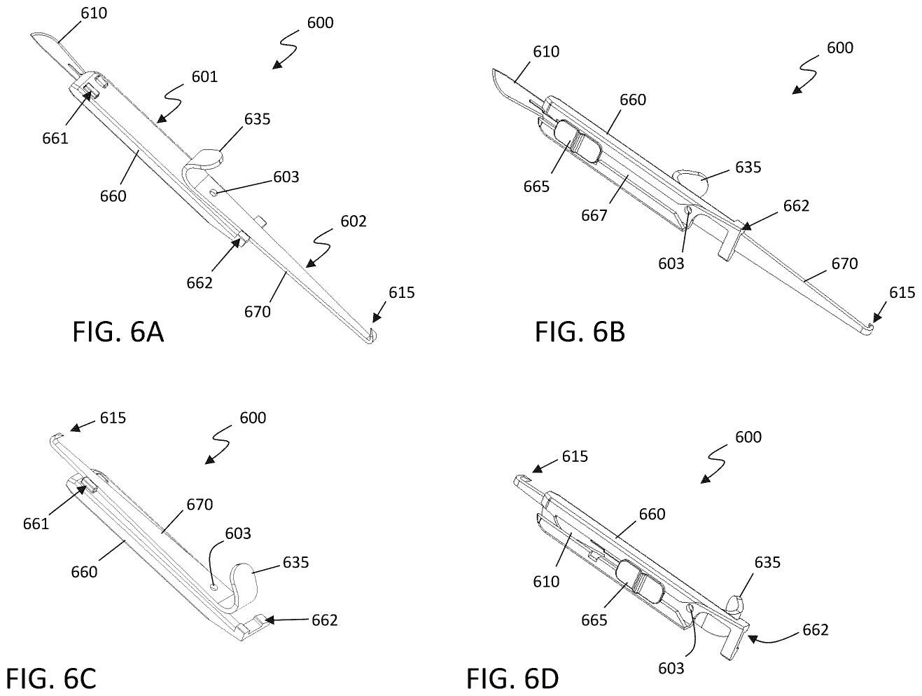

FIGS. 6A-6D show a cricothyrotomy device 600 in accordance with aspects of the present invention. In embodiments, the device 600 includes a scalpel portion 601 and a tracheal hook portion 602 that are pivotally connected to one another, e.g., by a pivoting joint 603 such as a pin joint. In embodiments, the scalpel portion 601 includes a blade 610 (which may be similar of similar construction and function as blade 110) and the tracheal hook portion 602 includes a tracheal hook 615 (which may be similar of similar construction and function as tracheal hook 115). Each of the scalpel portion 601 and the tracheal hook portion 602 may include one or more gripping features such as surface texturing, one or more grip tabs, etc., e.g., as described herein. For example, as shown in FIGS. 6A-6D, the tracheal hook portion 602 may comprise a grip tab 635. The scalpel portion 601 may include a blade cover (which may be similar of similar construction and function as blade cover 140).

In accordance with aspects of the present invention, the scalpel portion 601 includes a body 660, a first locking mechanism 661 at a first end of the body 660, and a second locking mechanism 662 at a second end of the body 660 opposite the first end of the body 660. In embodiments, the scalpel portion 601 includes a slider 665 fixedly connected to the blade 610 and slidably connected to the body 660. In embodiments, the body 660 defines a cavity 667 that is configured to house the blade 610 when the blade 610 is in a retracted position. In embodiments, the blade 610 may be selectively retracted into the cavity 667 by moving the slider 665 in a first direction relative to the body 660 and may be selectively extended out of the cavity by moving the slider 665 in a second direction relative to the body 660 opposite the first direction. In this manner, a user may selectively move the blade 610 between a first (retracted) position and a second (extended) position via manipulation of the slider 665.

In accordance with aspects of the present invention, the tracheal hook portion 602 includes a body 670, the tracheal hook 615 at a first end of the body 670, and the grip tab 635 at a second end of the body 670 opposite the first end of the body 670. In embodiments, the body 660 of the scalpel portion 601 and the body 670 of the tracheal hook portion 602 are pivotally connected to one another by the pivoting joint 603. In the example implementation depicted in FIGS. 6A-6D, the pivoting joint 603 is closer to the grip tab 635 at the second end of the body 670 than it is to the tracheal hook 615 at the first end of the body 670, and the pivoting joint 603 is closer to the second locking mechanism 662 at the second end of the body 660 than it is to the first locking mechanism 661 at the first end of the body 660. This arrangement provides a compact (e.g., shorter) overall shape when the tracheal hook portion 602 is rotated to a first (e.g., folded) position relative to the scalpel portion 601 as shown in FIGS. 6C and 6D, and an elongated (e.g., longer) overall shape when the tracheal hook portion 602 is rotated to a second (e.g., unfolded) position relative to the scalpel portion 601 as shown in FIGS. 6A and 6B.

In embodiments, a first portion of the body 670 is configured to engage the first locking mechanism 661 when the tracheal hook portion 602 is rotated to the first (e.g., folded) position relative to the scalpel portion 601 as shown in FIGS. 6C and 6D. In various configurations, the engagement is a friction fit or a snap fit that securely holds the device 600 in the folded position but that may be overcome by a force applied by a user to disengage the first portion of the body 670 from the first locking mechanism 661.

In embodiments, a second portion of the body 670 is configured to engage the second locking mechanism 662 when the tracheal hook portion 602 is rotated to the second (e.g., unfolded) position relative to the scalpel portion 601 as shown in FIGS. 6A and 6B. In various configurations, the engagement is a friction fit or a snap fit that securely holds the device 600 in the unfolded position but that may be overcome by a force applied by a user to disengage the second portion of the body 670 from the second locking mechanism 662.

Additional aspects of the invention include manufacturing and/or using a cricothyrotomy device as described herein. Even further aspects of the invention include providing instructions for using a cricothyrotomy device as described herein. The instructions may be at least one of printed and video.

In an exemplary method of using a cricothyrotomy device in accordance with aspects of the present invention (e.g., device 100, 110′, 100″, 400, 500, or 600), a user (e.g., a medical professional) will begin by determining the patient's need for the cricothyrotomy procedure and locate the cricothyroid membrane on the patient's throat. After removing the scalpel cover from the scalpel blade (if a blade cover is present), the user will grip the device and use the blade (e.g., blade 110, 110′, 110″, 410, 510, or 610) to make the required incision through the skin and tissue into the trachea.

After articulating and reversing the device in the user's hand, the user will grip and utilize the tracheal hook (e.g., tracheal hook 115, 115′, 115″, 415, 515, or 615) to manipulate the opening and tissue before inserting the airway tubing. The tracheal hook is used to pull on the patient's tracheal ring to broaden the opening.

The airway tubing will be inserted into the incision opening to a sufficient depth to secure the artificial air passage but not overly deep to enter either of the patient's lung passages thereby providing ventilation to both lungs with one airway tube. Once inserted the patient's tissues will seal around the airway tube and the patient may be ventilated thereby. If desired and available, a carbon dioxide sensor will then be placed on or near the end of the airway tube to verify the exhalation of a specified concentration of carbon dioxide from the patient's lungs.

The medical professional may then sterilize and clean the device (e.g., device 100, 110′, 100″, 400, 500, or 600) as necessary. In embodiments, the device is non-toxic and can be quickly and easily rinsed, washed, or sanitized for repeated reuse in mass casualty situations. The scalpel blade cover (if present) may then be articulated and latched back into its original position to protect the scalpel blade between uses. In a mass casualty situation, the device may then be reused on another patient by repeating the procedure.

All attachment and latching means may be made by all the methods and from all materials known to one of ordinary skill in the art to secure the components to their mating parts as needed.

The above-described components may be made from any material now or later known to one of ordinary skill in the art including, without limitation, plastic, metal, glass, ceramic, rubber, or any combination of a variety of different materials. Materials may be selected based on the application from criteria such as weight, corrosion resistance, strength, biocompatibility, and permeability, among others. All component parts may be manufactured by all known methods to one of skill in the art and in certain embodiments single components may be made from more than one subcomponent rigidly connected to other subcomponents.

Since numerous changes and modifications can be made in the above-described details without departing from the spirit and nature of the invention. It is to be understood that all such changes and modifications are included within the scope of the invention. Different embodiments of the invention may be constructed in a variety of lengths, diameters, sizes, styles, geometries, or using any number of components. Different embodiments may include different materials, coatings and surface textures known to one of ordinary skill in the art as appropriate to vary functionality. Variations of the embodiments may be constructed to accommodate different users and environments.

It is noted that the foregoing examples have been provided merely for the purpose of explanation and are in no way to be construed as limiting of implementations of the present invention. While aspects of the present invention have been described with reference to an exemplary embodiment, it is understood that the words which have been used herein are words of description and illustration, rather than words of limitation. Changes may be made, within the purview of the appended claims, as presently stated and as amended, without departing from the scope and spirit of the present disclosure in its aspects. Although implementations of the present invention have been described herein with reference to particular means, materials and embodiments, implementations of the present invention are not intended to be limited to the particulars disclosed herein; rather, implementations of the present invention extend to all functionally equivalent structures, methods and uses, such as are within the scope of the appended claims.

As used herein, the recitation of “at least one of A, B, or C” is intended to mean “either A alone, B alone, C alone, or any combination of two or more of A, B, and C.” Similarly, the recitation of “one or more of A, B, or C” is intended to mean “either A alone, B alone, C alone, or any combination of two or more of A, B, and C.”

Features which are described in the context of separate aspects and embodiments of the invention may be used together and/or be interchangeable unless the context clearly dictates otherwise. Similarly, features described in the context of a single embodiment may also be provided separately or in any suitable sub-combination unless the context clearly dictates otherwise.

What is claimed:

1. A cricothyrotomy device, comprising:
a scalpel portion comprising a blade at a first end of the device; and
a tracheal hook portion comprising a tracheal hook at a second end of the device opposite the first end of the device.

2. The cricothyrotomy device of claim 1, wherein:
the scalpel portion and the tracheal hook portion are comprised in a body of the device;
the blade is at a first end of the body; and
the tracheal hook is at a second end of the body opposite the first end of the body.

3. The cricothyrotomy device of claim 2, wherein the body includes one or more gripping features.

4. The cricothyrotomy device of claim 3, wherein the one or more gripping features comprise one or more of surface texturing on the body, a bend in the body, or one or more grip tabs extending outward from the body.

5. The cricothyrotomy device of claim 1, wherein the scalpel portion and the tracheal hook portion are releasably connected to one another.

6. The cricothyrotomy device of claim 1, wherein the scalpel portion and the tracheal hook portion are releasably connected to one another by one or more resilient clips.

7. The cricothyrotomy device of claim 6, wherein a flat surface of the tracheal hook portion abuts a flat surface of the scalpel portion when the scalpel portion and the tracheal hook portion are connected to one another.

8. The cricothyrotomy device of claim 6, wherein a grip tab of the tracheal hook portion abuts a grip tab of the scalpel portion when the scalpel portion and the tracheal hook portion are connected to one another.

9. The cricothyrotomy device of claim 1, wherein the scalpel portion and the tracheal hook portion are releasably connected to one another by a locking mechanism.

10. The cricothyrotomy device of claim 9, wherein the locking mechanism comprises:
a first portion configured to hold the tracheal hook portion;
a second portion configured to hold the scalpel portion; and
a hinge connecting the first portion and the second portion.

11. The cricothyrotomy device of claim 10, wherein the hinge comprises a living hinge.

12. The cricothyrotomy device of claim 10, wherein the locking mechanism is structured and arranged such that the first portion and the second portion are releasably connectable to one another by a snap mechanism that engages when the first portion and the second portion are pressed against one another.

13. The cricothyrotomy device of claim 10, wherein one of the first portion or the second portion includes a wedge that creates a friction lock amongst the scalpel portion, the tracheal hook portion, and the locking mechanism when the first portion and the second portion are pressed against one another to engage the snap mechanism.

14. The cricothyrotomy device of claim 1, wherein the scalpel portion and the tracheal hook portion are pivotally connected to one another.

15. The cricothyrotomy device of claim 14, wherein the scalpel portion and the tracheal hook portion are pivotally connected to one another by a pin joint.

16. The cricothyrotomy device of claim 14, wherein:
the scalpel portion comprises a body having a cavity; and
the blade is structured and arranged to be selectively retracted into the cavity and selectively extended out of the cavity.

17. The cricothyrotomy device of claim 16, wherein:
the scalpel portion comprises a slider connected to the blade; and
the scalpel portion is structured and arranged such that moving the slider in a first direction relative to the body retracts the blade into the cavity and moving the slider in a second direction relative to the body extends the blade out of the cavity.

18. A method comprising performing a cricothyrotomy procedure on a patient using the cricothyrotomy device of claim 1.

19. The method of claim 18, wherein the performing the cricothyrotomy procedure on the patient comprises:
making an incision in a trachea of the patient using the blade; and
broadening an incision opening created by the incision by pulling on a tracheal ring of the patient using the tracheal hook.

20. The method of claim 19, wherein the performing the cricothyrotomy procedure on the patient further comprises:
inserting an airway tubing into the incision opening.
